(19) United States
(12) Patent Application Publication
(10) Pub. No.: US 2012/0095315 A1
(43) Pub. Date: Apr. 19, 2012

(54) CONFIGURATION OF BLOOD GLUCOSE METER INTERFACES

(75) Inventors: James D. Tenbarge, Fishers, IN (US); Joseph M. Simpson, Fishers, IN (US); Kurt Klem, Indianapolis, IN (US); Blaine E. Ramey, Indianapolis, IN (US)

(73) Assignee: ROCHE DIAGNOSTICS OPERATIONS, INC., Indianapolis, IN (US)

(21) Appl. No.: 12/905,491

(22) Filed: Oct. 15, 2010

(51) Int. Cl. A61B 5/145

(52) U.S. Cl. 600/365

(57) ABSTRACT

A handheld diabetes management device includes a blood glucose measurement engine that measures a blood glucose level of a patient. The device includes a physical port that is exposed at an exterior of the device. The device includes a processing module that includes first, second, and third physical interfaces that are internal to the device. The device includes a multiplexing module that alternatively connects the physical port to one of the first, second, and third physical interfaces of the processing module. The processing module selectively operates the first physical interface using first and second modes. When an external host is connected to the physical port, the processing module selectively transfers information based on the blood glucose level to the external host using the first mode. When the external host is connected to the physical port, the processing module selectively provides file access to the external host using the second mode.

### FIELD

The present disclosure relates generally to handheld medical devices and more particularly to interface configuration for handheld blood glucose management devices.

### BACKGROUND

There is a need for a handheld diabetes management device offering a wide variety of interconnectivity options without sacrificing functionality. Often referred to just as diabetes, diabetes mellitus is a chronic condition in which a person has elevated blood glucose levels that result from defects in the body's ability to produce and/or use insulin. There are three main types of diabetes. Type 1 diabetes usually strikes children and young adults, and can be autoimmune, genetic, and/or environmental. Type 2 diabetes accounts for 90-95% of diabetes cases and is linked to obesity and physical inactivity. Gestational diabetes is a form of glucose intolerance diagnosed during pregnancy and usually resolves spontaneously after delivery.

In 2009, according to the World Health Organization, at least 220 million people worldwide suffer from diabetes. In 2005, an estimated 1.1 million people died from diabetes. Its incidence is increasing rapidly, and it is estimated that between 2005 and 2030, the number of deaths from diabetes will double. In the United States, nearly 24 million Americans have diabetes, with an estimated 25 percent of seniors age 60 and older being affected. The Centers for Disease Control and Prevention forecast that 1 in 3 Americans born after 2000 will develop diabetes during their lifetime. The National Diabetes Information Clearinghouse estimates that diabetes costs $132 billion in the United States alone every year. Without treatment, diabetes can lead to severe complications such as heart disease, stroke, blindness, kidney failure, amputations, and death related to pneumonia and flu.

Diabetes is managed primarily by controlling the level of glucose in the bloodstream. This level is dynamic and complex, and is affected by multiple factors including the amount and type of food consumed, and the amount of insulin (which mediates transport of glucose across cell membranes) in the blood. Blood glucose levels are also sensitive to exercise, sleep, stress, smoking, travel, illness, menses, and other psychological and lifestyle factors unique to individual patients. The dynamic nature of blood glucose and insulin, and all other factors affecting blood glucose, often require a person with diabetes to forecast blood glucose levels. Therefore, therapy in the form of insulin or oral medications, or both, can be timed to maintain blood glucose levels in an appropriate range.

Management of diabetes is time-consuming for patients because of the need to consistently obtain reliable diagnostic information, follow prescribed therapy, and manage lifestyle on a daily basis. Diagnostic information, such blood glucose, is typically obtained from a capillary blood sample with a lancing device and is then measured with a handheld blood glucose meter. Interstitial glucose levels may be obtained from a continuous glucose sensor worn on the body. Prescribed therapies may include insulin, oral medications, or both. Insulin can be delivered with a syringe, an ambulatory infusion pump, or a combination of both. With insulin therapy, determining the amount of insulin to be injected can require forecasting meal composition of fat, carbohydrates and proteins along with effects of exercise or other physiologic states. The management of lifestyle factors such as body weight, diet, and exercise can significantly influence the type and effectiveness of a therapy.

Management of diabetes involves large amounts of diagnostic data and prescriptive data acquired in a variety of ways: from medical devices, from personal healthcare devices, from patient-recorded logs, from laboratory tests, and from healthcare professional recommendations. Medical devices include patient-owned bG meters, continuous glucose monitors, ambulatory insulin infusion pumps, diabetes analysis software, and diabetes device configuration software. Each of these systems generates and/or manages large amounts of diagnostic and prescriptive data. Personal healthcare devices include weight scales, blood pressure cuffs, exercise machines, thermometers, and weight management software. Patient recorded logs include information relating to meals, exercise and lifestyle. Lab test results include HbA1C, cholesterol, triglycerides, and glucose tolerance. Healthcare professional recommendations include prescriptions, diets, test plans, and other information relating to the patient's treatment.

There is a need for a handheld patient device to aggregate, manipulate, manage, present, and communicate diagnostic data and prescriptive data from medical devices, personal healthcare devices, patient recorded information, biomarker information, and recorded information in an efficient manner to improve the care and health of a person with diabetes, so the person with diabetes can lead a full life and reduce the risk of complications from diabetes.

Consequently, there is a need for a handheld patient device that offers connectivity with a wide range of other devices, including healthcare devices, computers, consumer electronics, and accessories. There exists a need for a handheld patient device that serves as a hub for a patient's diabetes management, from glucose monitoring to insulin infusion to historical tracking. There exists a need for such a handheld patient device so that patients and clinicians will have more information to monitor and manage diabetes, thereby making diabetes management less intrusive and more appealing to the patient.

### SUMMARY

The present disclosure describes a handheld diabetes management device to provide interconnection options while minimizing a number of physical ports. The handheld diabetes management device includes a blood glucose measurement engine configured to measure blood glucose of a patient and a universal serial bus (USB) port including electrical conductors. The handheld diabetes management device also includes a USB control module, a serial control module that implements a second serial bus protocol, an audio amplifier module, and a multiplexing module electrically connected to the USB port.

The multiplexing module alternatively electrically connects ones of the electrical conductors of the USB port to one of the USB control module, the serial control module, and the audio amplifier. The USB control module is configured to selectively (i) operate using a personal healthcare device class (PHDC) based on information from a host connected to the USB port and (ii) operate using a mass storage class (MSC) based on the information. The handheld diabetes management device also includes a core processing module that communicates data based on the blood glucose measurement to the host via the USB port when the USB control module is operating using the PHDC.

A handheld diabetes management device for providing communication options while minimizing a number of physical connectors includes a blood glucose measurement engine that measures a blood glucose level of a patient. The handheld diabetes management device includes a physical port that includes electrical conductors and that is exposed at an exterior of the handheld diabetes management device. The handheld diabetes management device includes a processing module that provides a user interface to the patient and that includes first, second, and third physical interfaces that are internal to the handheld diabetes management device.

The handheld diabetes management device further includes a multiplexing module that is electrically connected to the physical port and that alternatively electrically connects ones of the electrical conductors to one of the first, second, and third physical interfaces of the processing module. The processing module selectively operates the first physical interface using first and second modes. When an external host is connected to the physical port, the processing module selectively transfers information based on the blood glucose level to the external host using the first mode of the first physical interface. When the external host is connected to the physical port, the processing module selectively provides file access to the external host using the second mode of the first physical interface.

Further areas of applicability of the present disclosure will become apparent from the detailed description, the claims and the drawings. The detailed description and specific examples are intended for purposes of illustration only and are not intended to limit the scope of the disclosure.

### BRIEF DESCRIPTION OF THE DRAWINGS

The present disclosure will become more fully understood from the detailed description and the accompanying drawings, wherein:

FIG. 1 shows a patient and a treating clinician;

FIG. 2 shows a patient with a continuous glucose monitor (CGM), an ambulatory insulin infusion pump, and a handheld diabetes management device;

FIG. 3 shows a diabetes management system used by patients and clinicians to manage diabetes;

FIG. 4 is a functional block diagram of an example implementation of a handheld diabetes management device; and

FIG. 5 is a flowchart of example operation of a handheld diabetes management device.

### DESCRIPTION

Systems and devices according to the present disclosure allow for a wide variety of interconnection options while minimizing the number of physical ports. Increasing the number of physical ports can lead to confusion regarding which physical port is associated with any given cable. Additional physical ports can also increase the number of associated cables and adapters necessary when using a device. With respect to safety, physical ports present a site for intrusion of foreign material, such as fluids. Further, cleanliness standards, including those established by regulatory bodies such as the Food and Drug Administration (FDA), can require the ability to clean a medical device. Systems and devices according to the present disclosure avoid these problems by multiplexing at least one physical port so that the physical port can accommodate multiple interface protocols. In addition, the present disclosure describes, for at least one of the interface protocols, interfacing with multiple classes of device using the same interface protocol. The present disclosure also describes leveraging wireless interfaces to allow for further interconnection options.

The following description is merely illustrative in nature and is in no way intended to limit the disclosure, its application, or uses. For purposes of clarity, the same reference numbers will be used in the drawings to identify similar elements. As used herein, the phrase at least one of A, B, and C should be construed to mean a logical (A or B or C), using a non-exclusive logical OR. It should be understood that steps within a method can be executed in different order without altering the principles of the present disclosure.

As used herein, the term module can refer to, be part of, or include an Application Specific Integrated Circuit (ASIC); an electronic circuit; a combinational logic circuit; a field programmable gate array (FPGA); a processor (shared, dedicated, or group) that executes code; other suitable components that provide the described functionality; or a combination of some or all of the above, such as in a system-on-chip. The term module can include memory (shared, dedicated, or group) that stores code executed by the processor.

The term code, as used above, can include software, firmware, and/or microcode, and can refer to programs, routines, functions, classes, and/or objects. The term shared, as used above, means that some or all code from multiple modules can be executed using a single (shared) processor. In addition, some or all code from multiple modules can be stored by a single (shared) memory. The term group, as used above, means that some or all code from a single module can be executed using a group of processors. In addition, some or all code from a single module can be stored using a group of memories.

The apparatuses and methods described herein can be implemented by one or more computer programs executed by one or more processors. The computer programs include processor-executable instructions that are stored on a non-transitory tangible computer readable medium. The computer programs can also include stored data. Non-limiting examples of the non-transitory tangible computer readable medium are nonvolatile memory, magnetic storage, and optical storage.

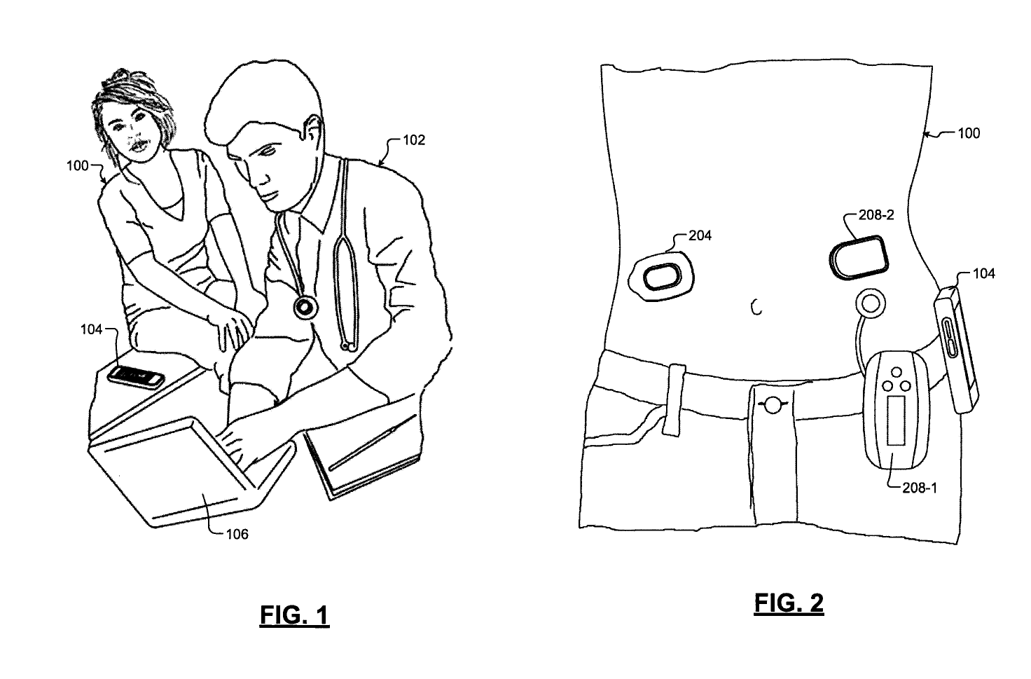

Referring now to FIG. 1, a patient 100 with diabetes and a clinician 102 are shown in a clinic environment. The term ‘patient’ encompasses persons with metabolic syndrome, pre-diabetes, type 1 diabetics, type 2 diabetics, and gestational diabetics. The term ‘clinician’ is used broadly to include nurses, nurse practitioners, physicians, endocrinologists, etc.

During a healthcare consultation, the patient 100 typically shares with the clinician 102 a variety of patient data including blood glucose measurements, continuous glucose monitor data, amounts of insulin infused, amounts of food and beverages consumed, exercise schedules, and other lifestyle information. The clinician 102 can obtain additional patient data that includes measurements of HbA1C, cholesterol levels, triglycerides, blood pressure, and weight. The patient data can be recorded manually and/or can be recorded electronically on a handheld diabetes management device 104, diabetes analysis software executed on a computing device such as a personal computer (PC) 106, and/or a web-based diabetes analysis site (not shown). The term PC, as used herein, includes computers using a Microsoft operating system as well as computers using an Apple operating system, Linux, OpenBSD, Ubuntu, etc.

The clinician 102 can analyze the patient data manually and/or can analyze the patient data electronically using the diabetes analysis software and/or the web-based diabetes analysis site. After analyzing the patient data and reviewing adherence of the patient 100 to previously prescribed therapy, the clinician 102 can decide whether to modify the therapy for the patient 100.

Referring now to FIG. 2, the patient 100 can use a continuous glucose monitor (CGM) 204, an ambulatory durable insulin pump 208-1, an ambulatory non-durable insulin pump 208-2, and the handheld diabetes management device 104. In various implementations, the durable insulin pump 208-1 and the non-durable insulin pump 208-2 can be used interchangeably, and the handheld diabetes management device 104 can be configured to interact with whichever of the insulin pumps 208-1 or 208-2 is currently in use. In various implementations, if both the insulin pumps 208-1 and 208-2 are worn by the patient 100, the handheld diabetes management device 104 can communicate with only one of the insulin pumps 208-1 or 208-2. In various other implementations, the handheld diabetes management device 104 can communicate with both of the insulin pumps 208-1 and 208-2. The insulin pumps 208-1 and 208-2 will be referred to collectively herein as insulin pump 208.

The CGM 204 uses a subcutaneous sensor to sense and monitor the amount of glucose in the blood of the patient 100 and communicates corresponding readings to the handheld diabetes management device 104. The handheld diabetes management device 104 performs various tasks including measuring and recording blood glucose levels, determining an amount of insulin to be administered to the patient 100 via the insulin pump 208, receiving patient data via a user interface, archiving the patient data, etc. When the CGM 204 is in use, the handheld diabetes management device 104 periodically receives readings from the CGM 204 indicating glucose level in the blood of the patient 100. When the insulin pump 208 is in use, the handheld diabetes management device 104 transmits instructions to the insulin pump 208, which delivers insulin to the patient 100. Insulin can be delivered in a scheduled manner at a basal rate, which attempts to maintain a predetermined insulin level in the blood of the patient 100. Additionally, insulin can be delivered in the form of a bolus dose, which raises the amount of insulin in the blood of the patient 100 by a predetermined amount.

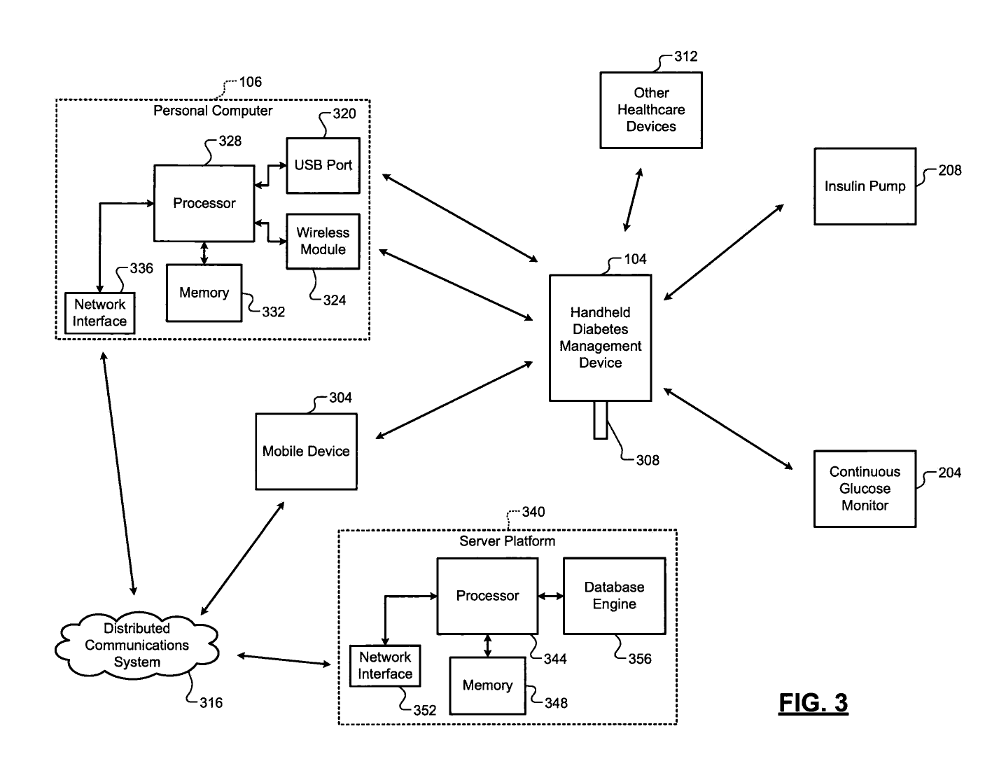

Referring now to FIG. 3, a diabetes management system used by the patient 100 and the clinician 102 includes one or more of the following devices: the handheld diabetes management device 104, the CGM 204, the insulin pump 208, the PC 106 with the diabetes analysis software, a mobile device 304, and other healthcare devices represented collectively at 312. The handheld diabetes management device 104 is configured as a system hub and communicates with the devices of the diabetes management system. Alternatively, the insulin pump 208 or the mobile device 304 can be configured as the system hub. Communication between the devices in the diabetes management system can be performed using wireless interfaces (e.g., Bluetooth) and/or wireline interfaces (e.g., USB). For example, one of versions 1.1, 1.2, 2.0, 2.1, 3.0, and 4.0 of the Bluetooth standard can be used.

Communication protocols used by these devices can include protocols compliant with the IEEE 11073 standard, which can be extended using guidelines provided by Continua® Health Alliance Design Guidelines. For example, IEEE 11703-20601 Optimized Exchange Protocol with the IEEE 11073-10417 Blood Glucose Device Specialization standards can be used. In addition, the device specialization can be supplemented by predefined Roche proprietary communications protocols, which for example can include additional measurement objects. Further, healthcare records systems such as Microsoft® HealthVault™ and Google™ Health can be used by the patient 100 and/or the clinician 102 to exchange information.

The handheld diabetes management device 104 can receive blood glucose readings from one or more sources, such as the CGM 204. The CGM 204 continuously measures the blood glucose level of the patient 100. The CGM 204 periodically communicates the blood glucose level to the handheld diabetes management device 104. In various implementations, the handheld diabetes management device 104 and the CGM 204 communicate wirelessly.

Additionally, the handheld diabetes management device 104 includes blood glucose meter (BGM) functionality. The handheld diabetes management device 104 can measure glucose levels from a sample from the patient 100. For example, in various implementations, the handheld diabetes management device 104 can receive a blood glucose measurement strip 308. The patient 100 deposits a sample of blood or other bodily fluid on the blood glucose measurement strip 308. The handheld diabetes management device 104 analyzes the sample to determine the blood glucose level in the sample. The blood glucose level measured from the sample and/or the blood glucose level read by the CGM 204 can be used in determining the amount of insulin to be administered to the patient 100.

The handheld diabetes management device 104 communicates with the insulin pump 208. The insulin pump 208 can be configured to receive instructions from the handheld diabetes management device 104 to deliver a predetermined amount of insulin to the patient 100. Additionally, the insulin pump 208 can receive additional information including meal and/or exercise schedules of the patient 100. In various implementations, the insulin pump 208 can determine the amount of insulin to administer based on the additional information.

The insulin pump 208 can also communicate data to the handheld diabetes management device 104. The data can include amounts of insulin delivered to the patient 100, corresponding times of delivery, and pump status. In various implementations, the handheld diabetes management device 104 and the insulin pump 208 can communicate wirelessly.

In addition, the handheld diabetes management device 104 can communicate with the other healthcare devices 312. For example, the other healthcare devices 312 can include a blood pressure meter, a weight scale, a pedometer, a fingertip pulse oximeter, a thermometer, etc. The other healthcare devices 312 obtain and communicate personal health information of the patient 100 to the handheld diabetes management device 104 through wireless, USB, or other interfaces. The other healthcare devices 312 can use communication protocols compliant with ISO/IEEE 11073 extended using guidelines from Continual® Health Alliance. Further, the devices of the diabetes management system can communicate with each other via the handheld diabetes management device 104.

The handheld diabetes management device 104 can communicate with the PC 106 using Bluetooth, USB, or other interfaces. Diabetes management software running on the PC 106 includes an analyzer-configurator that stores configuration information of the devices of the diabetes management system. The configurator has a database to store configuration information of the handheld diabetes management device 104 and the other devices. The configurator can communicate with users through web pages or computer screens in non-web applications. The configurator transmits user-approved configurations to the devices of the diabetes management system. The analyzer retrieves data from the handheld diabetes management device 104, stores the data in a database, and outputs analysis results through standard web pages or computer screens in non-web based applications.

The handheld diabetes management device 104 can communicate with the mobile device 304 using wired or wireless protocols, such as Bluetooth. Examples of the mobile device 304 include a cellular phone, a pager, a personal digital assistant (PDA), a tablet computing device, etc. The mobile device 304 can communicate with a network, such as a distributed communications system 316. In various implementations, the distributed communications system 316 can be the Internet. The handheld diabetes management device 104 can send and receive messages, including data and instructions, to the distributed communications system 316 via the mobile device 304.

The PC 106 includes a USB port 320 and/or a wireless module 324. A processor 328 controls communications over the USB port 320 and/or the wireless module 324. The processor 328 can execute instructions from memory 332. Further, the PC 106 includes a network interface 336, which can be wired, such as Ethernet, or wireless, such as WiFi (including 802.11a, b, g and/or n). The network interface 336 can communicate with the distributed communications system 316. The PC 106 can therefore also serve as an intermediary between the distributed communications system 316 and the handheld diabetes management device 104. The PC 106 and the handheld diabetes management device 104 can communicate using USB and using a wireless protocol, such as Bluetooth. In various implementations, the wireless protocol can be a network protocol, such as WiFi. In such implementations, functionality of the wireless module 324 and the network interface 336 can be combined into a single module.

When the handheld diabetes management device 104 is connected via USB to the PC 106, the handheld diabetes management device 104 can charge an internal power supply, such as a rechargeable battery. The PC 106 can be replaced by any other device having sufficient processing capability, such as a laptop, a netbook, or a tablet computing device. The PC 106 can execute software stored in nonvolatile storage of the PC 106. For example only, some or all of the memory 332 can be nonvolatile. Additionally or alternatively, other nonvolatile storage media can be present, such as flash memory, magnetic storage, and optical storage.

The PC 106 can execute specialized software corresponding to the handheld diabetes management device 104 and can execute general purpose software that can interact with the handheld diabetes management device 104. The PC 106 can also execute software provided by the handheld diabetes management device 104. The software provided by the handheld diabetes management device 104 can then be stored persistently on the PC 106 or can be removed when the PC 106 is no longer in communication with the handheld diabetes management device 104. In various implementations, the software provided by the handheld diabetes management device 104 can be low- or zero-footprint software such that when the handheld diabetes management device 104 is no longer in communication with the PC 106, traces of the software, such as files and settings, are not left behind on the PC 106.

The PC 106 can also acquire software via the distributed communications system 316, such as from a server platform 340. For example, the server platform 340 can provide web server functionality. The PC 106 can download and execute software from the server platform 340. In various implementations, the downloaded software can be web-based, such as a Java or Flash application. The local applications can communicate data and instructions with the server platform 340. In addition, the PC 106 can interact with a server-side application executed by the server platform 340. The remote and local applications may be known collectively as Accu-Chek 360°.

The applications can have varying functionality and access authorizations. For example, some applications can be authorized to access historical data from the handheld diabetes management device 104, while other applications can be authorized to control operation of the handheld diabetes management device 104, such as glucose measurement settings and insulin pump settings. In various implementations, the applications can also control other devices, such as the insulin pump 208 and the CGM 204, via the handheld diabetes management device 104. For example, the applications can update firmware, retrieve configuration settings and error codes, and provide configuration settings. The applications can also control firmware updates of the handheld diabetes management device 104 itself.

The server platform 340 can include one or more physical servers having multiple processors, but logically includes at least a processor 344 that executes instructions from memory 348 and communicates with the distributed communications system 316 via a network interface 352. Further, the processor 344 communicates with a database engine 356, which can be executed by a separate processor and memory and/or by the processor 344 itself, such as in a virtual machine.

The database engine 356 stores one or more databases, which can track firmware versions of the handheld diabetes management device 104 as well as associated devices, such as the CGM 204 and the insulin pump 208. The database engine 356 can store contact information for the patient 100 so that, for example, firmware updates and other alerts can be communicated to the patient 100. The database engine 356 can also store historical data from the handheld diabetes management device 104. The stored data can be used for remote access by the patient 100, the clinician 102, or in the case of a failure or erasure of the handheld diabetes management device 104.

The database engine 356 can also store language settings and localizations for various regions, and can track which of these languages are installed in the handheld diabetes management device 104. The database engine 356 can also store food and exercise databases that indicate the corresponding effect on blood sugar of various foods and activities. These databases can be supplemented by data entered by the patient 100 and/or the clinician 102 via the handheld diabetes management device 104 or via some other interface such as one presented by the PC 106. The database engine 356 can also store user preferences for the handheld diabetes management device 104 as well as treatment parameters for the handheld diabetes management device 104, such as equations and/or constants for calculating amounts of insulin.

A number of device classes are defined for use with the USB protocol. In various implementations, the handheld diabetes management device 104 implements the mass storage device class (MSC) and the personal healthcare device class (PHDC). In various implementations, the handheld diabetes management device 104 also implements the communications device class (CDC) and/or the remote network driver interface specification (RNDIS).

The MSC is used to access raw data, such as files and file systems. For example only, USB flash drives and memory card readers for digital cameras and video-cameras generally implement the MSC. The MSC is supported by a wide variety of operating systems, including Microsoft Windows, which has offered native support for MSC since Windows 2000. In various implementations, the handheld diabetes management device 104 can also implement the media transfer protocol (MTP), which also allows file access.

In various implementations, the handheld diabetes management device 104 stores blood glucose data, insulin data, exercise data, and food data as files that can be accessed via the MSC. The handheld diabetes management device 104 can also store software for use by the PC 106. In addition, the handheld diabetes management device 104 can store documentation, such as health files, frequently asked questions files, and training videos and podcasts.

The handheld diabetes management device 104 can store web pages and other interactive content. For example only, the handheld diabetes management device 104 can store a start webpage, from which a user can access other information and options. The handheld diabetes management device 104 can be configured so that the start webpage or a startup program is automatically executed when the handheld diabetes management device 104 is connected to an appropriately configured computer. The startup program can provide the option of installing the necessary components for PHDC support. To allow access via the MSC, the handheld diabetes management device 104 can implement a FAT32 file system or another file system, such as FAT, HFS Plus, and Ext2.

The PC 106 may not natively support the PHDC. For example, drivers and/or configuration files may be required prior to the PC 106 supporting communication with the handheld diabetes management device 104 using the PHDC. The handheld diabetes management device 104 can therefore store various drivers and configuration files to support the PHDC operation for one or more operating systems. The PHDC was designed to allow interoperability between medical devices, and can support IEEE 11073 operation. The PC 106 can communicate with the handheld diabetes management device 104 using the PHDC in order to obtain medical data, such as blood glucose readings and historical insulin injection records. The PC 106 can also use the PHDC to read and command basal rate and bolus parameters.

The handheld diabetes management device 104 can implement the CDC to allow direct communication between the PC 106 and various components of the handheld diabetes management device 104. The CDC allows a variety of pre-existing communication protocols, such as serial protocols and network protocols, to be carried over USB. For example only, the PC 106 can communicate with an internal component of the handheld diabetes management device 104 using the CDC. Additionally, the PC 106 can use the CDC to communicate with other devices, such as the CGM 204 and the insulin pump 208, via the handheld diabetes management device 104. In addition, the CDC can be used during manufacturing, testing, calibration, and repair. For example, the CDC can be used by a specialized test environment, such as a test stand, and/or by a computer having specialized software. In various implementations, end users are prevented from using the CDC. Additionally or alternatively to implementing the CDC, the Remote Network Driver Interface Specification (RNDIS) can be used. RNDIS is a specification for supporting network devices over USB, and is supported natively in some Microsoft operating systems.

As discussed above, the handheld diabetes management device 104 can communicate with the PC using a wireless protocol such as Bluetooth. Although Bluetooth is described herein for purposes of illustration only, other protocols can be used, such as ZigBee or Bluetooth low energy. Similar to the classes of USB, profiles are defined for Bluetooth. For example, the handheld diabetes management device 104 can implement a serial port profile (SPP) and/or a health device profile (HDP). The SPP defines protocols and procedures to allow devices to emulate a serial protocol, such as RS-232, using Bluetooth.

In various implementations, the SPP can be used in similar scenarios as the CDC of USB. For example, the SPP can be used by the PC 106 to communicate with the CGM 204 and/or the insulin pump 208 via the handheld diabetes management device 104. Further, the handheld diabetes management device 104 can use the SPP to communicate with the CGM 204 and the insulin pump 208. For example only, the SPP can be used for configuration and updating of the CGM 204 and the insulin pump 208. The handheld diabetes management device 104 can use the HDP for supplying and receiving medical data to and from the CGM 204 and the insulin pump 208, such as blood glucose readings and insulin doses.

The HDP can be used in conjunction with IEEE 11073. The HDP can be used when transmitting medical information from the handheld diabetes management device 104 to the PC 106. The medical information can include glucose readings, exercise data, and food data from handheld diabetes management device 104 as well as glucose readings from the CGM 204 and insulin dosing history from the insulin pump 208.

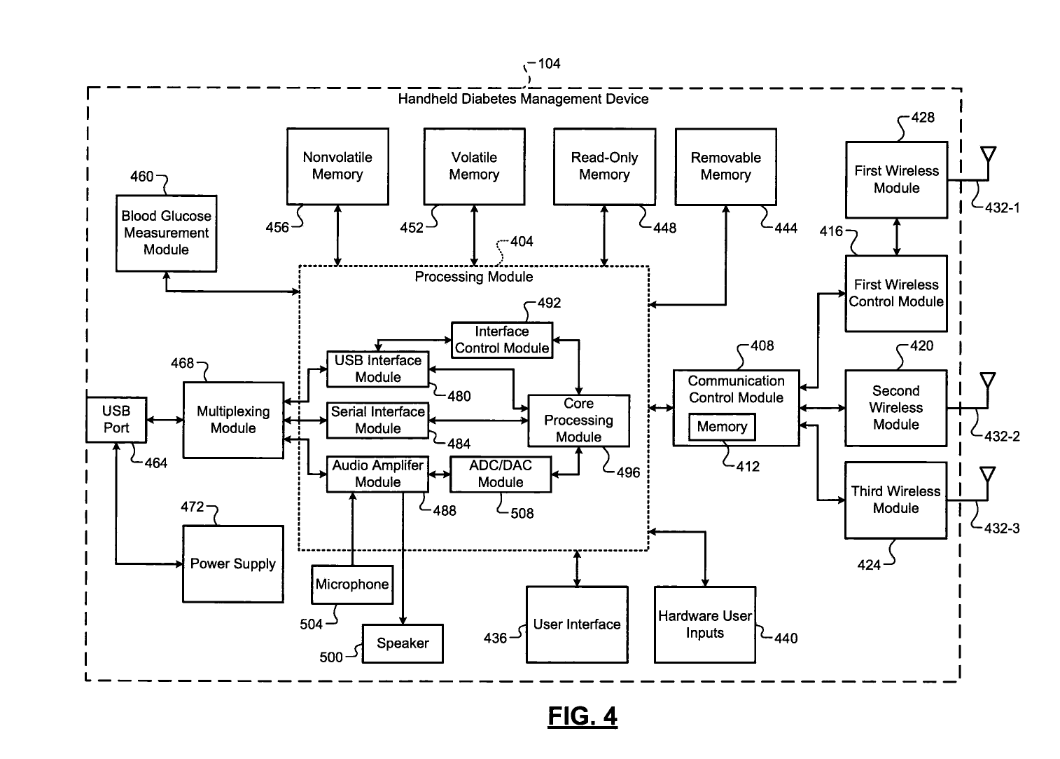

Referring now to FIG. 4, a functional block diagram of an example implementation of the handheld diabetes management device 104 is presented. The handheld diabetes management device 104 includes a processing module 404, such as the i.MX233 applications processor from Freescale Semiconductor, Inc. The processing module 404 communicates with a communication control module 408, such as an STM32F103 32-bit ARM Cortex microcontroller unit from ST Microelectronics. For example only, the communication control module 408 includes memory 412 having volatile and nonvolatile components. For example only, the communication control module 408 includes 512 KB of flash memory and 64 KB of random access memory (RAM).

The processing module 404 and the communication control module 408 can communicate using universal asynchronous receiver/transmitters (UARTs). In various implementations, a level shifter or a voltage transformer is interposed between the processing module 404 and the communication control module 408 to match signal levels of the respective UARTs. The communication control module 408 controls wireless communication. In various implementations, the communication control module 408 controls a first wireless control module 416 and a second wireless module 420. In various implementations, the communication control module 408 also controls a third wireless module 424.

The first wireless control module 416 controls a first wireless module 428, which can implement RF processing and/or baseband processing. Antennas 432-1, 432-2, and 432-3 are illustrated as being connected to the first wireless module 428, the second wireless module 420, and the third wireless module 424, respectively. However, more or fewer antennas can be used. When fewer antennas are used, access to the antenna can be multiplexed and/or different frequency operating ranges can allow antenna to be used by different modules simultaneously. Further, RF and/or baseband processing can be shared between modules. In various implementations, the first wireless control module 416 can subsume the functionality of the first wireless module 428.

The first wireless control module 416 can implement encryption, such as the advanced encryption standard (AES), to prevent eavesdropping and other malicious activity from affecting the wireless communication. The first wireless control module 416 can implement a proprietary wireless protocol operating in a specified frequency band, such as the 2.4 GHz industrial, scientific, and medical (ISM) band. For example, the first wireless control module 416 can be an nRF24LE1™ ultra-low-power wireless system-on-chip solution from Nordic Semiconductor, Inc.

The second wireless module 420 can implement a wireless personal area network (WPAN) protocol such as Bluetooth, Bluetooth low energy, or Zigbee. For example only, the second wireless module 420 can be a BL6450 controller from Texas Instruments. When present, the third wireless module 424 can implement another proprietary wireless protocol and/or a wireless local area network (WLAN) protocol, such as IEEE 802.11(a, b, g, and/or n).

The processing module 404 communicates with a user interface 436, which can include a liquid crystal display (LCD) touchscreen, which can be backlit by light-emitting diodes (LEDs). For example only, the touchscreen can be a WQVGA (400×240) 3-inch screen. The processing module 404 can receive hardware user inputs 440. For example only, the hardware user inputs can include buttons and switches, such as a microswitch for performing a hardware reset. The microswitch can be recessed to prevent accidental actuation.

The processing module 404 can communicate with removable memory 444, such as flash storage, including secure digital (SD), compact flash (CF), and other flash storage technologies. For example, the removable memory 444 can be a microSD card. The removable memory 444 can be used to store the software, instructional material, and drivers to be provided to the PC 106 and/or the mobile device 304.

The processing module 404 also communicates with read-only memory 448. The read-only memory 448 can include an electrically erasable programmable read-only memory (EEPROM). The processing module 404 communicates with volatile memory 452, such as synchronous dynamic random access memory (SDRAM). The processing module 404 communicates with nonvolatile memory 456, such as NAND flash memory. In various implementations, some or all of the read-only memory 448, the volatile memory 452, and the nonvolatile memory 456 can be incorporated on the same die or in the same package as the processing module 404.

The processing module 404 communicates with a blood glucose measurement module 460, which analyzes a sample from the patient 100 to determine a glucose level in the patient's blood. For example only, the blood glucose measurement module 460 can dispense test strips to which a blood sample is applied. In various implementations, the blood glucose measurement module 460 processes readings from the sample and provide a blood glucose number to the processing module 404. Alternatively, the blood glucose measurement module 460 can provide raw data to the processing module 404, which determines a blood glucose level based on the raw data.

The handheld diabetes management device 104 includes a USB port 464. For example, the USB port 464 can be a standard USB port, a micro USB port, or a mini USB port. Specifically, the USB port 464 can be a micro-B female port. The small size of the micro-B port offers less area for potential fluid or other contaminant intrusion, and allows a physical size of the handheld diabetes management device 104 to be minimized.

To reduce the number of physical ports required in the handheld diabetes management device 104, a multiplexing module 468, such as an MC34825 from Freescale Semiconductor, Inc., is connected to the USB port 464. The multiplexing module 468 can allow the USB port 464 to be used for USB purposes, for a non-USB serial interface, and for an audio interface. In addition, a power supply 472 can be connected to the USB port 464. The power supply 472 can include a battery, such as a lithium ion rechargeable battery, which provides power to components of the handheld diabetes management device 104.

The power supply 472 can be recharged via the USB port 464. In various implementations, the power supply 472 can be recharged when the USB port 464 is connected to the PC 106 and/or a powered USB hub. In addition, a separate adapter can be used to recharge the power supply 472 via the USB port 464. In various implementations, the current required by the power supply 472 is greater than can be provided by a computer, and charging therefore requires the charging adapter. The charging adapter can be integrated with a stand that retains the handheld diabetes management device 104 and also allows interaction with the user interface 436 of the handheld diabetes management device 104. Charging using the USB port 464 allows a separate charging port to be eliminated.

In various implementations, the multiplexing module 468 can analyze cables and/or devices connected to the USB port 464. For example only, termination resistances, pull-up resistances, and pull-down resistances of cables and/or devices attached to the USB port 464 can indicate to the multiplexing module 468 the type of device connected to the USB port 464. When the multiplexing module 468 determines that a USB device is attached, the multiplexing module 468 can connect the USB port 464 to a USB interface module 480 of the processing module 404.

Similarly, the multiplexing module 468 can connect the USB port 464 to a serial interface module 484 when a non-USB serial device is determined to be connected to the USB port 464. The multiplexing module 468 connects the USB port 464 to an audio amplifier module 488 when an audio device is determined to be connected to the USB port 464.

The USB interface module 480 is controlled by an interface control module 492, which determines whether the USB interface module 480 should be operating using the PHDC, the MSC, the CDC, or another class. The interface control module 492 communicates with a core processing module 496, which can coordinate operation of the processing module 404 and also implement user interface features. In various implementations, the core processing module can run Windows CE from Microsoft Corp.

After manufacturing, the interface control module 492 can initially instruct the USB interface module 480 to operate using the CDC. This mode can be used for programming, configuration, calibration, and testing. The interface control module 492 can then control the USB interface module 480 to operate using the MSC prior to providing the handheld diabetes management device 104 to the patient 100. Repair and testing facilities can provide signals and/or commands to the interface control module 492 to indicate that CDC is once again required, such as if the handheld diabetes management device 104 is returned for servicing.

When the handheld diabetes management device 104 is with the patient 100, the interface control module 492 can set the USB interface module 480 to use the MSC by default. However, if it is determined that the device connected to the USB port 464 has the necessary drivers and configuration to use the PHDC, the interface control module 492 can set the USB interface module 480 to use the PHDC. A user of the handheld diabetes management device 104 and user of the connected device can override this operation and cause the interface control module 492 to maintain the USB interface module 480 using the MSC. The MSC can allow the USB interface module 480 to provide access to the removable memory 444. When operating using the CDC, the USB interface module 480 can communicate with, for example, the blood glucose measurement module 460 and/or with the communication control module 408.

The serial interface module 484 implements a non-USB protocol, such as the inter-integrated circuit (I²C) protocol, General Purpose Input/Output (GPIO), and/or RS-232. The non-USB serial protocol can be used during manufacturing and testing by test equipment that has a serial interface. By multiplexing the non-USB pins onto the USB port 464, the need for a separate serial port, such as a 9-pin DE-9, can be eliminated.

The audio amplifier module 488 can include an audio amplifier for powering a speaker 500 and an audio amplifier for amplifying signals from a microphone 504. An ADC/DAC module 508 converts analog microphone signals into digital and converts digital sound signals into analog signals for playback. The ADC/DAC module 508 communicates with the core processing module 496. For example only, the microphone 504 can be used to record journal entries corresponding to insulin doses, exercise, meals, and blood glucose readings. The speaker 500 can be used to play back journal entries and/or to play audio and video files, such as instructional videos. The audio and video files can be stored in the removable memory 444.

The audio amplifier module 488 is selectively connected to the USB port 464 by the multiplexing module 468. The pins of the USB port 464 are thereby used as microphone and/or headphone conductors. By using the USB port 464, the need for separate audio connectors, such as tip/ring/sleeve (TRS) connectors, can be eliminated. An adapter can be used that simply electrically connects one or two TRS connectors to the conductors on a USB plug. When the adapter is used, standard headphones and microphones can be used. When an audio device, such as a set of headphones, with or without a microphone, is connected to the USB port 464, the audio amplifier module 488 can disable amplification of the microphone 504 and the speaker 500.

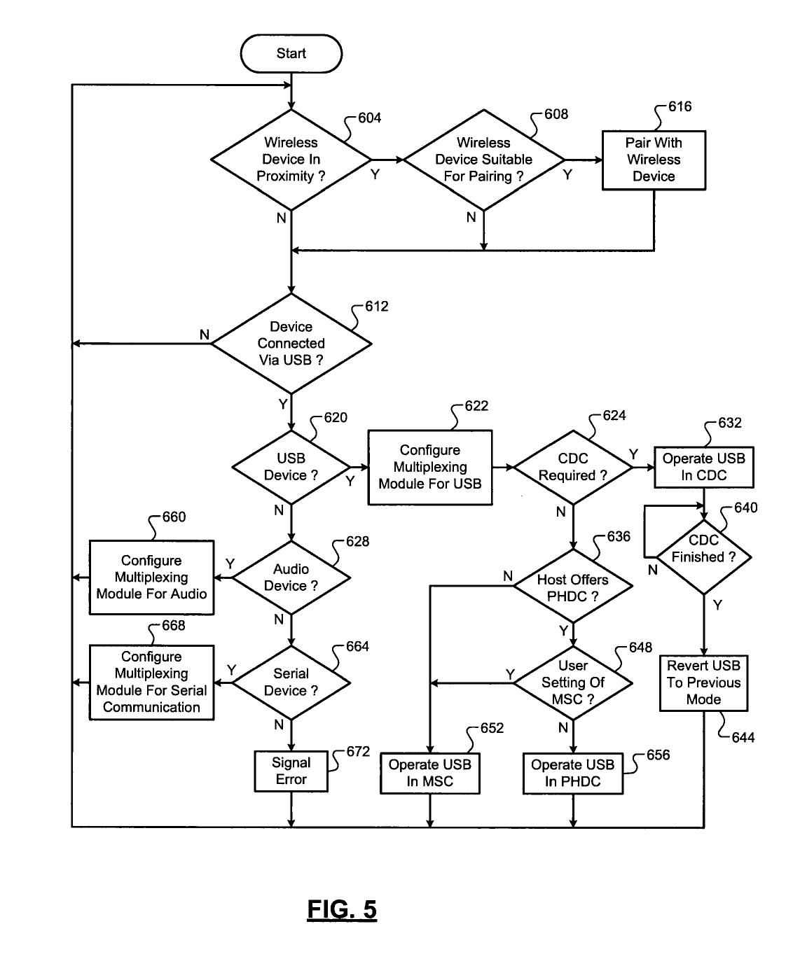

Referring now to FIG. 5, a flowchart of example operation of the handheld diabetes management device 104 is presented. Control begins at 604, where control determines whether a wireless device is in proximity to the handheld diabetes management device 104. For example, this wireless device can be a Bluetooth device or a device implementing the same proprietary protocol as the first wireless control module 416. If there is a wireless device in proximity, control transfers to 608; otherwise, control transfers to 612.

At 608, control determines whether the wireless device is suitable for pairing or otherwise interfacing with. If so, control transfers to 616; otherwise, control transfers to 612. Wireless devices that are suitable for pairing/interfacing can be specified by manufacturer, device type, and/or authentication codes. For example, pairing with a Bluetooth headset can be disallowed while pairing with an insulin pump is allowed. Compatibility between the insulin pump and the handheld diabetes management device 104 can be determined before or after pairing. If compatibility is determined after pairing, pairings with incompatible devices can later be terminated. At 616, control pairs with the suitable wireless device, such as a continuous glucose monitor or an insulin pump. Additionally, pairing can be performed with the PC 106 and/or the mobile device 304. Control continues at 612.

At 612, control determines whether a device is connected via the USB port. If so, control transfers to 620; otherwise, control returns to 604. At 620, control determines whether the connected device is a USB device. If so, control transfers to 622; otherwise, control transfers to 628. At 622, control configures the multiplexing module for USB operation and continues at 624. The connected USB device can be referred to as a host.

At 624, control determines whether the CDC is required or requested. If so, control transfers to 632; otherwise, control transfers to 636. For example, the CDC can be required by a test or manufacturing interface and/or a diagnostic/troubleshooting application on a personal computer. At 632, control switches operation of the USB to the CDC. Control continues at 640. Control remains at 640 until use of the CDC is finished, at which point control transfers to 644. Control determines that use of the CDC is finished when a corresponding indication is received from the host, when a user of the handheld diabetes management device 104 requests that the CDC operation end, or when the host is disconnected. At 644, control reverts USB operation to the previous mode (such as the MSC or the PHDC) and returns to 604.

At 636, control determines whether the host offers PHDC. If so, control transfers to 648; otherwise, control transfers to 652. At 652, control causes the USB to operate using the MSC. Control then returns to 604. While operating in the MSC, the host can obtain firmware and/or configuration files that allow the host to offer PHDC. Once PHDC operation is enabled, when control again arrives at 636, control would then transfer to 648. At 648, control determines whether a user setting has requested operation using the MSC. If so, control transfers to 652; otherwise, control transfers to 656. At 656, control operates USB using the PHDC and returns to 604.

At 628, control determines whether the attached device is an audio device. If so, control transfers to 660; otherwise, control transfers to 664. At 660, control configures the multiplexing module for audio and returns to 604. At 664, control determines whether a non-USB serial device is connected. If so, control transfers to 668; otherwise, control transfers to 672. At 668, control configures the multiplexing module for non-USB serial communication and returns to 604. At 672, the type of the connected device does not appear to be one of the recognized devices, and therefore an error is signaled and control returns to 604.

The present disclosure describes a handheld diabetes management device to provide interconnection options while minimizing a number of physical ports. The handheld diabetes management device includes a blood glucose measurement engine configured to measure blood glucose of a patient and a universal serial bus (USB) port including electrical conductors. The handheld diabetes management device also includes a USB control module, a serial control module that implements a second serial bus protocol, an audio amplifier module, and a multiplexing module electrically connected to the USB port.

The multiplexing module alternatively electrically connects ones of the electrical conductors of the USB port to one of the USB control module, the serial control module, and the audio amplifier. The USB control module is configured to selectively (i) operate using a personal healthcare device class (PHDC) based on information from a host connected to the USB port and (ii) operate using a mass storage class (MSC) based on the information. The handheld diabetes management device also includes a core processing module that communicates data based on the blood glucose measurement to the host via the USB port when the USB control module is operating using the PHDC.

In other features, the handheld diabetes management device further includes a processing module that includes the core processing module, the audio amplifier module, the serial control module, and the USB control module. The second serial bus protocol is based on a universal asynchronous receiver/transmitter (UART). The second serial bus protocol is RS-232. The multiplexing module selects one of the USB control module, the serial control module, and the audio amplifier based on characteristics of a cable connected to the USB port. The USB port is one of a female mini USB port, a female micro USB port, and a female standard USB port.

In further features, the USB control module is further configured to operate using a communication device class (CDC) based on a request from the host. The USB control module prevents operation using the CDC when the host is a computer of the patient. The USB control module operates using Remote Network Driver Interface Specification (RNDIS) based on a request from the host. The USB control module prevents operation using the RNDIS when the host is a computer of the patient.

In still other features, the handheld diabetes management device further includes a wireless communications module that selectively establishes communication with a continuous glucose monitor (CGM) using a proprietary industrial, scientific, and medical (ISM) band wireless interface. The handheld diabetes management device further includes a second wireless communications module that selectively establishes communication with an insulin pump using a Bluetooth wireless interface.

A handheld diabetes management device for providing communication options while minimizing a number of physical connectors includes a blood glucose measurement engine that measures a blood glucose level of a patient. The handheld diabetes management device includes a physical port that includes electrical conductors and that is exposed at an exterior of the handheld diabetes management device. The handheld diabetes management device includes a processing module that provides a user interface to the patient and that includes first, second, and third physical interfaces that are internal to the handheld diabetes management device.

The handheld diabetes management device further includes a multiplexing module that is electrically connected to the physical port and that alternatively electrically connects ones of the electrical conductors to one of the first, second, and third physical interfaces of the processing module. The processing module selectively operates the first physical interface using first and second modes. When an external host is connected to the physical port, the processing module selectively transfers information based on the blood glucose level to the external host using the first mode of the first physical interface. When the external host is connected to the physical port, the processing module selectively provides file access to the external host using the second mode of the first physical interface.

In other features, the physical port is a Universal Serial Bus (USB) port. The first physical interface is a USB interface. The first mode is a personal healthcare device class (PHDC). The second mode is a mass storage device class (MSC). The processing module operates using the second mode when the external host does not offer the first mode. The processing module also selectively operates the first physical interface using a third mode. The third mode is a communications device class (CDC).

In further features, the second and third physical interfaces are a non-USB serial interface and an audio interface, respectively. The handheld diabetes management device further includes first and second wireless interfaces that implement first and second wireless protocols, respectively; and a communication control module that is in communication with the processing module and that controls the first and second wireless interfaces. The communication control module operates the first wireless interface in first and second wireless modes.

In still other features, the first wireless protocol is Bluetooth. The first and second wireless modes are a health device profile (HDP) and a serial port profile (SPP), respectively. The second wireless protocol is a 2.4 GHz wireless interface. The handheld diabetes management device further includes a third wireless interface that implements a third wireless protocol.

What is claimed is:

1. A handheld diabetes management device to provide interconnection options while minimizing a number of physical ports, the handheld diabetes management device comprising:
a blood glucose measurement engine configured to measure blood glucose of a patient;
a universal serial bus (USB) port including electrical conductors;
a USB control module;
a serial control module that implements a second serial bus protocol;
an audio amplifier module;
a multiplexing module electrically connected to the USB port,
wherein the multiplexing module alternatively electrically connects ones of the electrical conductors of the USB port to one of the USB control module, the serial control module, and the audio amplifier, and
wherein the USB control module is configured to selectively (i) operate using a personal healthcare device class (PHDC) based on information from a host connected to the USB port and (ii) operate using a mass storage class (MSC) based on the information; and
a core processing module that communicates data based on the blood glucose measurement to the host via the USB port when the USB control module is operating using the PHDC.

2. The handheld diabetes management device of claim 1 further comprising a processing module that includes the core processing module, the audio amplifier module, the serial control module, and the USB control module.

3. The handheld diabetes management device of claim 1 wherein the second serial bus protocol is based on a universal asynchronous receiver/transmitter (UART).

4. The handheld diabetes management device of claim 3 wherein the second serial bus protocol is RS-232.

5. The handheld diabetes management device of claim 1 wherein the multiplexing module selects one of the USB control module, the serial control module, and the audio amplifier based on characteristics of a cable connected to the USB port.

6. The handheld diabetes management device of claim 1 wherein the USB port is one of a female mini USB port, a female micro USB port, and a female standard USB port.

7. The handheld diabetes management device of claim 1 wherein the USB control module is further configured to operate using a communication device class (CDC) based on a request from the host, and wherein the USB control module prevents operation using the CDC when the host is a computer of the patient.

8. The handheld diabetes management device of claim 1 wherein the USB control module operates using Remote Network Driver Interface Specification (RNDIS) based on a request from the host, and wherein the USB control module prevents operation using the RNDIS when the host is a computer of the patient.

9. The handheld diabetes management device of claim 1 further comprising a wireless communications module that selectively establishes communication with a continuous glucose monitor (CGM) using a proprietary industrial, scientific, and medical (ISM) band wireless interface.

10. The handheld diabetes management device of claim 9 further comprising a second wireless communications module that selectively establishes communication with an insulin pump using a Bluetooth wireless interface.

11. A handheld diabetes management device for providing communication options while minimizing a number of physical connectors, the handheld diabetes management device comprising:
a blood glucose measurement engine that measures a blood glucose level of a patient;
a physical port that includes electrical conductors and that is exposed at an exterior of the handheld diabetes management device;
a processing module that provides a user interface to the patient and that includes first, second, and third physical interfaces that are internal to the handheld diabetes management device; and
a multiplexing module that is electrically connected to the physical port and that alternatively electrically connects ones of the electrical conductors to one of the first, second, and third physical interfaces of the processing module,
wherein the processing module selectively operates the first physical interface using first and second modes,
wherein when an external host is connected to the physical port, the processing module selectively transfers information based on the blood glucose level to the external host using the first mode of the first physical interface,
wherein when the external host is connected to the physical port, the processing module selectively provides file access to the external host using the second mode of the first physical interface.

12. The handheld diabetes management device of claim 11 wherein the physical port is a Universal Serial Bus (USB) port.

13. The handheld diabetes management device of claim 12 wherein the first physical interface is a USB interface, and wherein the first mode is a personal healthcare device class (PHDC).

14. The handheld diabetes management device of claim 12 wherein the second mode is a mass storage device class (MSC), wherein the processing module operates using the second mode when the external host does not offer the first mode.

15. The handheld diabetes management device of claim 14 wherein the processing module also selectively operates the first physical interface using a third mode, wherein the third mode is a communications device class (CDC).

16. The handheld diabetes management device of claim 11 wherein the second and third physical interfaces are a non-USB serial interface and an audio interface, respectively.

17. The handheld diabetes management device of claim 11 further comprising:
first and second wireless interfaces that implement first and second wireless protocols, respectively; and
a communication control module that is in communication with the processing module and that controls the first and second wireless interfaces,
wherein the communication control module operates the first wireless interface in first and second wireless modes.

18. The handheld diabetes management device of claim 17 wherein the first wireless protocol is Bluetooth, and wherein the first and second wireless modes are a health device profile (HDP) and a serial port profile (SPP), respectively.

19. The handheld diabetes management device of claim 18 wherein the second wireless protocol is a 2.4 GHz wireless interface.

20. The handheld diabetes management device of claim 17 further comprising a third wireless interface that implements a third wireless protocol.
